(19) United States
(12) Patent Application Publication
(10) Pub. No.: US 2007/0105139 A1
(43) Pub. Date: May 10, 2007

(54) Fluorescent probe and fluorescence detecting method

(75) Inventors: Junji Nishigaki, Kanagawa (JP); Hiroyuki Hirai, Kanagawa (JP); Naoki Sugimoto, Hyogo-ken (JP); Daisuke Miyoshi, Hyogo-ken (JP)

Correspondence Address: BIRCH STEWART KOLASCH & BIRCH, PO BOX 747, FALLS CHURCH, VA, 22040-0747, US

(73) Assignee: FUJIFILM Corporation, Tokyo (JP)

(21) Appl. No.: 11/543,037

(22) Filed: Oct. 5, 2006

(30) Foreign Application Priority Data
Oct. 7, 2005 (JP) 2005-295246

(51) Int. Cl. C12Q 1/68; C07H 21/04

(52) U.S. Cl. 435/6; 536/25.32; 977/924

(57) ABSTRACT

A fluorescent probe having a base sequence complementary to a specific sequence in a target nucleic acid, wherein the fluorescent probe has one end labeled with a nano particle fluorescent material, and the other end labeled with a fluorescent dye capable of fluorescence resonance energy transfer from the nano particle fluorescent material.

### CROSS-REFERENCE TO RELATED APPLICATION

This application claims priority under 35 USC 119 from Japanese patent Application No. 2005-295246, the disclosure of which is incorporated by reference herein.

### BACKGROUND OF THE INVENTION

1. Field of the Invention

The invention relates to a fluorescent probe and a fluorescence detecting method, and more particularly to a fluorescent probe having a base sequence complementary to a specific sequence in a target nucleic acid, and a fluorescence detecting method for detecting the specific sequence in the target nucleic acid by using the same.

2. Description of the Related Art

Various methods have been reported so far about detection of specific variation for the purpose of diagnosis of various diseases caused by defect in base sequence due to mutation in a gene, such as tumor or genetic disease.

For example, in Electrophoresis, 1995, vol. 16, No. 1, pp. 8-10, a method is reported in which the difference of the higher-order structure of a single chain caused by difference in base composition due to defects in base sequence is detected in terms of an electrophoretic pattern.

In Nature Biotechnology, 1996 (March) vol. 14, pp. 303-308, a method is reported about real-time monitoring of gene amplifying reaction using a so-called molecular beacon (composed of a probe complementary to the target, and mutually complementary arms connected to its both ends). A fluorescent dye and a quenching dye are bonded to both ends of the probe including the probe complementary to the target and the mutually complementary arms bonded to its both ends.

The principle of the monitoring method using the molecular beacon is as follows. When the molecular beacon exists alone, both arms at both ends form intramolecular duplex, so that dyes approach each other, and the fluorescence of the fluorescent dye is quenched by resonance energy transfer. On the other hand, when the target nucleic acid is present, the probe and the target form duplex while breaking the duplex in the probe, so that the distance between dyes increases to suppress the resonance energy transfer. As a result, the probe emits light. The presence of the target can be detected by measuring the emission.

The dyes used in the molecular beacon have an excitation wavelength that is close to the fluorescent wavelength. Therefore, an excitation light cut filter is necessarily attached to a fluorescence detector in order to reduce the noise derived from the excitation light. However, the excitation light cut filter also cuts off the fluorescent signal, thus having a fundamental problem of sensitivity loss.

To avoid this problem, an attempt has been proposed (see, for example, Japanese Patent Application Laid-Open (JP-A) No. 10-127300) in which the wavelength separation of excitation light from the fluorescent signal is improved by using fluorescence resonance energy transfer (FRET) between the two fluorescent dyes.

However, generally employed laser excitation promotes light fading of the dye. Therefore, detection using a conventional molecular beacon often has problems in quantifiability and reproducibility, and the problems have not been solved even by a system using the FRET disclosed in JP-A No. 10-127300.

### SUMMARY OF THE INVENTION

When using a molecular beacon, as described above, the sensitivity may be lowered by the excitation light cut filter used for reducing the noise, and if the FRET is used, the quantifiability and reproducibility may be impaired by photodecomposition (light fading) of the dye caused by laser excitation.

The present invention has been made in consideration of the above problems, and provides a fluorescent probe and a fluorescence detecting method.

It is hence an object of the invention to present a fluorescent probe capable of detecting a specific nucleic acid sequence with high quantifiability and reproducibility without loss of sensitivity, and a nucleic acid sequence detecting method using the same.

A first aspect of the invention is to provide (1) A fluorescent probe having a base sequence complementary to a specific sequence in a target nucleic acid, the fluorescent probe having one end labeled with a nano particle fluorescent material, and the other end labeled with a fluorescent dye capable of fluorescence resonance energy transfer from the nano particle fluorescent material.

A second aspect of the invention is to provide a fluorescence detecting method for detecting a specific sequence in a target nucleic acid with a fluorescent probe having a base sequence complementary to a specific sequence in the target nucleic acid, the method comprising putting a fluorescent probe in a condition in which the fluorescent probe is allowed to interact with the target nucleic acid, irradiating excitation light for a nano particle fluorescent material, and measuring the light emission from the fluorescent probe. The fluorescent probe has one end labeled with the nano particle fluorescent material, and the other end labeled with a fluorescent dye capable of fluorescence resonance energy transfer from the nano particle fluorescent material.

In the fluorescent probe according to the invention, the fluorescent material labeling its one end is a nano particle fluorescent material, and this nano particle fluorescent material is capable of fluorescence resonance energy transfer to a fluorescent dye. By using such a nano particle fluorescent material, the separation of the emission from the excitation light is improved compared to when a general fluorescent dye is used. Therefore, the excitation light cut filter is not needed, and the sensitivity loss can be avoided. Moreover, since the donor that absorbs the strong excitation light is an inorganic particle, light fading of the fluorescent dye can be suppressed easily.

### BRIEF DESCRIPTION OF THE DRAWINGS

FIG. 1 is conceptual diagram showing the principle of the change in fluorescence intensity in the invention.

FIG. 2A shows the emission spectrum at the time a fully matching target DNA is hybridized with a fluorescent probe according to an embodiment of the invention (main graph), and the emission spectrum at the time a target DNA including mismatch is hybridized with the fluorescent probe (framed graph), and FIG. 2B shows changes in the fluorescence intensity at 560 nm.

### DETAILED DESCRIPTION OF THE INVENTION

The fluorescent probe according to the invention is a florescence probe having a base sequence complementary to a specific sequence in a target nucleic acid. One end of the fluorescent probe is labeled with a nano particle fluorescent material (donor nano particle fluorescent material), and the other end is labeled with a fluorescent dye (acceptor dye) capable of fluorescent resonant energy transfer from the nano particle fluorescent material.

The nucleic acid detecting method according to the invention comprises using a fluorescent probe having a base sequence complementary to a specific sequence in a target nucleic acid to detect the specific sequence in the target nucleic acid. One end of the fluorescent probe is labeled with a nano particle fluorescent material, and the other end is labeled with a fluorescent dye capable of fluorescence resonance energy transfer from the nano particle fluorescent material.

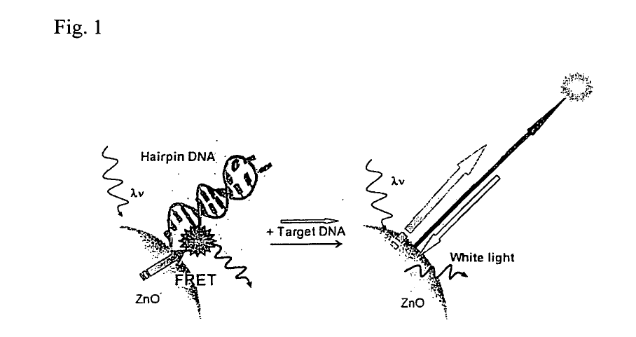

In the invention, the probe to be used for the detection of the base sequence has an oligonucleotide complementary to the base sequence of the target nucleic acid, the both ends of which are labeled respectively with a donor nano particle fluorescent material and an acceptor fluorescent dye. The donor nano particle fluorescent material and the acceptor fluorescent dye cause fluorescence resonance energy transfer (FRET). For example, in FIG. 1, zinc oxide is used as the donor nano particle fluorescent material.

When the probe exists alone, the probe is considered to take a curved and twisted shape due to heat fluctuation of the molecule or accidental formation of an intramolecular base pair. Hence, the dyes in the fluorescent probe molecule come close to each other, and fluorescence resonance energy transfer occurs. As a result, for example, when irradiated with an excitation light for the nano particle fluorescent material as the donor, the emission intensity (Y) at the maximum emission wavelength (Bnm) of the acceptor dye relative to the emission intensity (X) at the maximum emission wavelength (Anm) of the donor nano particle fluorescent material is higher. That is, the ratio, Y/X, increases (left illustration in FIG. 1).

When the fluorescent probe and the target nucleic acid exist together, base pairs are formed between the fluorescent probe and the target to form a duplex since the bases thereof are complementary. In the state of the duplex, the distance of the donor and the acceptor is longer than in the case of probe alone owing to the rigid double helical structure. Therefore, the FRET is lowered, and the Y/X value declines (right illustration in FIG. 1).

Accordingly, the presence or absence of the specific sequence or a point mutation can be detected from the FRET efficiency; i.e., the Y/X value.

Further, by measuring the difference in various different conditions, it can be detected more accurately and with high sensitivity whether abnormality such as base displacement, deletion, insertion or the like is included in the base sequence of the target.

In the present invention, one end of the probe is labeled with a nano particle fluorescent material. The nano particle fluorescent material can be excited at a wavelength in the ultraviolet region, and the fluorescence caused by the FRET appears in the visible region. Hence, there is no need for providing an excitation light cut filter or the like, and thus there is no sensitivity loss. As a result, the target nucleic acid having the specific sequence can be detected with high quantifiability and reproducibility.

The invention will be described in detail below.

<Fluorescent Probe>

The probe according to the invention has a base sequence (nucleotide) that is complementary to a specific sequence in a target nucleic acid, and a nano particle fluorescent material (a donor nano particle fluorescent material) is bonded to one end of the oligonucleotide, and a fluorescent dye (an acceptor dye) is bonded to the other end.

The combination of the nano particle fluorescent material (donor) and the acceptor dye is not particularly limited as far as fluorescence resonance energy transfer occurs.

[Nano Particle Fluorescent Material]

The nano particle fluorescent material may be any nano particle fluorescent material having a number-average particle diameter of a nano size. The number-average particle diameter is preferably 0.5 to 100 nm, more preferably 0.5 to 50 nm, and most preferably 1 to 10 nm, from the viewpoint of fluorescence intensity and affinity for biological molecules. The particle diameter distribution of the nano particle fluorescent material in terms of the coefficient of variation is preferably 0 to 50%, more preferably 0 to 20%, and most preferably 0 to 10%. The coefficient of variation is calculated by dividing the arithmetic standard deviation by the number-average particle diameter, and is expressed in percentage (arithmetic standard deviation×100/number-average particle diameter).

The nano particle fluorescent material as the donor is preferably a nano particle fluorescent material of a metal oxide or a metal sulfide, and examples of the metal contained in the metal oxide or metal sulfide include metals of the IIB group such as Zn, metals of the IIIA group such as Y, Eu, and Tb, metals of the IIIB group such as Ga and In, metals of the IVA group such as Zr and Hf, metals of the IVB group such as Si and Ge, metals of the VA group such as V and Nb, and metals of the VIA group such as Mo and W. Preferred examples thereof include: oxides of a metal selected from Y, Eu, Tb, Tm, Ba, Ca, Mg, Al, Mn, Zn, Si, Sr, Ga,Yb, Cr, Ce, Pb and W; ZnS; ZnSe; ZnTe; CdO; CdS; CdSe; CdTe; HgS; HgSe; HgTe; InP; InAs; GaN; GaP; GaAs; TiO₂; PbS; and PbSe. Among them, Zn is particularly preferred because Zn is mild to organisms. Composite metal oxides may be also used, such as Zn₂SiO₄, CaSiO₃, MgWO₄, YVO₄, and Y₂SiO₅. In particular, it is preferred to use a metal oxide, which is less toxic to human, and which emits fluorescence in a broad wavelength region as energy donor. ZnO is most preferred, for example because it can be manufactured stably, the particles thereof have superior monodispersibility, it emits intense radiation, and the wavelength region of the emission spectrum is suitable for the purpose.

The nano particle fluorescent material of the metal oxide or metal sulfide preferably contains a small amount of different metal ions from the metal of the metal oxide or metal sulfide. Examples of such metal ions include metal ions of Mn, Cu, Eu, Tb, Tm, Ce, Al, or Ag. These metal ions are preferably doped as compounds combined with chloride ions or fluoride ions. Ions of only one metal atom may be doped, or ions of two or more metal atoms may be doped. The concentration of such additional metal ion varies depending on the metal of the nano particle fluorescent material and the type of the additional metal ion, and is preferably 0.001 to 10 atom %, and more preferably 0.01 to 10 atom %.

The nano particle fluorescent material according to the invention is excited preferably by light in the ultraviolet region from the viewpoint of the separation of the excitation light and the signal fluorescence, usability of inexpensive light sources, and formation of a simple detection system mechanism. The nano particle fluorescent material is more preferably excited by an ultraviolet light having a wavelength of 250 to 380 nm. Upon receiving the ultraviolet excitation light, the nano particle fluorescent material emits light preferably in the visible region, more preferably a visible light within the range of 400 nm to 700 nm. The emitted visible light can excite the fluorescent dye in the visible region, whereby the emission from the fluorescent dye can be achieved with lower energy and reactivity without any violent reactivity to the human body. [Manufacturing Method of Nano Particle Fluorescent Material and its Dispersion Liquid]

A nano particle fluorescent material composed of a metal oxide can be obtained in various liquid phase synthesis methods, such as a sol-gel method of hydrolyzing an organic metal compound (such as alkoxide or acetyl acetonate) of the metal to be contained, a hydroxide precipitation method of adding alkali to an aqueous solution of a salt of the metal to precipitate the metal as a hydroxide followed by dehydrating and annealing, an ultrasonic decomposition method of emitting ultrasonic wave to the solution of the precursor of the metal, a solvothermal method of conducting a decomposition reaction at high temperature and high pressure, and a spray pyrolysis of spraying at high temperature. Other methods include vapor phase analysis methods such as thermal CVD method using an organic metal compound, a plasma CVD method, a sputtering method using a target made of the metal or an oxide of the metal, and a laser ablation method.

A nano particle fluorescent material composed of a metal sulfide can be obtained in various liquid phase synthesis methods, such as a hot soap method involving crystal growth of a pyrolytic metal compound, such as diethylthio carbamate compound, of the metal to be contained in a high boiling organic solvent, such as trialkyl phosphine oxide, trialkyl phosphine, or ω-amino alkane, a coprecipitation method involving crystal growth caused by addition of a solution of a sulfide such as sodium sulfide or ammonium sulfide to a solution of a salt of the metal, and a reverse micelle method of allowing the raw material aqueous solution containing a surfactant to exist as reverse micelle in a nonpolar organic solvent such as alkane, ether, or aromatic hydrocarbon, and allowing crystal growth in the reverse micelle. Similarly to the case of the nano particle fluorescent material composed of a metal oxide, the nano particle fluorescent material composed of a metal sulfide can be also obtained by vapor phase analysis methods.

[Surface Modifier of Nano Particle Fluorescent Material]

The nano particle fluorescent material according to the invention is preferably modified by a surface modifier. As a result, the dispersibility of the nano particle fluorescent material in water or hydrophilic solvent can be improved, and the elution and/or quenching of the nano particle fluorescent material caused by body fluid can be prevented. There is also advantage in that the connection to the molecular probe for detecting the target molecule gets easier.

The surface modifier used in the invention is a compound shown in formula (I) below or its decomposition product.
M-(R)₄ Formula (I)
In the formula, M represents a Si or Ti atom, and R represents an organic group. Rs may be the same as or different from each other, and at least one of Rs represents a group that is reactive to an affinity molecule.

In the organic group represented by R, the group reactive to an affinity molecule may be, for example, has, at its terminal, any one of a vinyl group, an allyloxy group, an acryloyl group, a methacryloyl group, an isocyanato group, a formyl group, an epoxy group, a maleimide group, a mercapto group, an amino group, a carboxyl group, or a halogen, and such a terminal group may be bonded to a connecting group L. Among these reactive groups, one having an amino group at its terminal is particularly preferred.

Examples of the connecting group L include alkylene groups, preferably chain or cyclic alkylene groups having 1 to 10 (more preferably 1 to 8) carbon atoms, such as a methylene group, an ethylene group, a trimethylene group, a tetramethylene group, a hexamethylene group, a propylene group, an ethyl ethylene group, or a cyclohexylene group.

The connecting group L may have an unsaturated bond. Examples of the unsaturated group include alkenylene groups (preferably chain or cyclic alkylene groups having 1 to 10 (more preferably 1 to 8) carbon atoms such as a vinylene group, a propenylene group, a 1-butenylene group, a 2-butenylene group, a 2-pentenylene group, a 8-hexadecenylene group, a 1,3-butane dienylene group, a cyclohexenylene group), arylene groups (preferably arylene groups having 6 to 10 (more preferably 6) carbon atoms such as a phenylene group, a naphthylene group, and a phenylene group).

The connecting group L may also have one or more hetero atoms, which mean any atoms other than a carbon atom, e.g., a nitrogen atom, an oxygen atom, a sulfur atom). The hetero atoms are each preferably an oxygen atom or a sulfur atom, and an oxygen atom is most preferred. The number of the hetero atoms is not limited, and is preferably 5 or smaller, or more preferably 3 or smaller.

The connecting group L may also contain a functional group having a carbon atom adjacent to the hetero atom as a partial structure. Examples of the functional group include ester groups (including carboxylic esters, carbonic esters, sulfonic esters, and sulfinic esters), amide groups (including carboxylic amides, urethanes, sulfonic amides, and sulfinic amides), ether groups, thioether groups, disulfide groups, amino groups, and imide groups. The functional group may further have a substituent, and L may have a plurality of such functional groups. When there are plural functional groups, they may be the same as or different from each other.

Preferable examples of the functional group include ester groups, amide groups, ether groups, thioether groups, disulfide groups, and amino groups. Alkenyl groups, ester groups, and ether groups are more preferable.

Other examples of the organic group represented by R include arbitrary groups, and preferred examples thereof are alkoxy groups and phenoxy groups such as a methoxy group, an ethoxy group, an isopropoxy group, an n-propoxy group, a t-butoxy group, an n-butoxy group. These alkoxy groups and phenoxy groups may further have a substituent, and the total number of the carbon atoms is preferably 8 or less.

The surface modifier used in the invention may have at least one of an amino group and a carboxyl group that forms a salt with acid or base.

When the surface modifier used in the invention is a decomposition product of a compound represented by formula (I), the decomposition product is a hydroxide formed by hydrolysis of an alkoxy group, a low molecular weight oligomer formed by dehydration condensation reaction between hydroxyl groups (the structure of the oligomer may be a linear structure, a cyclic structure, or a crosslinked structure), a condensation reaction product formed by dealcoholization reaction between a hydroxy group and an unhydrolyzed alkoxy group, or a sol or gel formed by dehydration of any of the above decomp osition products.

Specific examples of the surface modifier are listed below. These examples should not be construed as limiting the present invention:

N-(2-aminoethyl)-3-aminopropyl methyl dimethoxy silane,

N-(2-aminoethyl)-3-aminopropyl trimethoxy silane, N-(2-aminoethyl)-3-aminopropyl triethoxy silane, 3-aminopropyl trimethoxy silane, aminophenyl trimethoxy silane, 3-aminopropyl triethoxy silane, bis(trimethoxysilylpropyl) amine,

N-(3-aminopropyl)-benzamide trimethoxy silane, 3-hydrazide propyl trimethoxy silane, 3-maleimide propyl trimethoxy silane, (p-carboxy) phenyl trimethoxy silane, 3-carboxy propyl trimethoxy silane, 3-aminopropyl titanium tripropoxide, 3-aminopropyl methoxyethyl titanium diethoxide, and 3-carboxypropyl titanium trimethoxide.

The surface modifier used in the invention may have a terminal NH₂ or COOH group that forms a salt with acid or base. The surface modifier used in the invention may cover the entire surface of the nano particle fluorescent material or may be bonded to a part of the surface of the nano particle fluorescent material. In the invention, only one surface modifier may be used, or plural surface modifiers may be used in combination.

In addition to the surface modifier, other known surface modifiers (for example, polyethylene glycol, polyoxy ethylene (1) lauryl ether phosphoric acid, lauryl ether phosphoric acid, trioctyl phosphine, trioctyl phosphine oxide, sodium polyphosphate, sodium bis(2-ethylhexyl) sulfosuccinate) may be present at the time of the synthesis of the nano particles or after the synthesis of the nano particles.

In the explanation of the surface modifier represented by formula (I), the affinity molecule that can be bonded to R refers to a molecule that can be bonded to the nano particle fluorescent material via the surface of the surface modifier. The affinity molecule may be, for example, the nucleotide described later.

[Acceptor Dye]

The acceptor fluorescent dye used in the invention is not particularly limited, and the absorption maximum wavelength thereof is preferably 400 nm to 650 nm, more preferably 450 nm to 600 nm, or most preferably 500 nm to 600 nm, from the viewpoint of the distance from the excitation wavelength peak of the donor fluorescent dye and the efficiency of the energy transfer.

Examples of the acceptor dye include Cy3, Cy5, HEX, fluorescein, amino fluorescein, amino acetamide methyl fluorescein, amino acetamide fluorescein, Lucifer yellow, tetramethyl rhodamine, rhodamine X, Texas red, eosine, and erythrosine. The azamethine compounds disclosed in JP-A No. 2001-089482 are also usable. Among them, from the viewpoint of the distance from the excitation peak wavelength and the efficiency of the energy transfer, the acceptor dye is preferably Cy3, Cy5, HEX, rhodamine X, Texas red, or an azamethine compound. Cy3, Cy5, HEX, and an azamethine compound are particularly preferred.

[Nucleotide]

Nucleic acid (probe moiety) complementary to the specific sequence in the target nucleic acid can interact with a sample nucleic acid to form a double bond when coming close to the sample nucleic acid according to the degree of the complementarity.

The length of the nucleotide constituting the probe moiety is not particularly limited, and is generally 10 to 70, preferably 10 to 25, and more preferably 15 to 19. An excessively long probe is not preferred since the improvement of the detection sensitivity is not expected while the handling is more complicated. The oligonucleotide in the probe may have, at both ends, sequences not complementary to the specific sequence in the target. However, the presence of the extra sequences is not preferred because the extra sequences form base pairs with sequences other than the specific base sequence of the target. The oligonucleotide in the probe may be an oligonucleotide that is chemically modified (e.g., phosphorothioated) so as not to be decomposed by various nucleases.

The method for attaching the acceptor dye and the donor nano particle fluorescent material to the terminals of the oligonucleotide is, for example, a chemical synthesis method. It is not limited which of the acceptor dye or the donor nano particle fluorescent material is bonded to the 3′ terminal (or 5′ terminal) of the nucleotide sequence.

Specifically, the method may be selected from, but is not limited to, the following methods. The 3′ terminal can be labeled directly with the acceptor dye by using 3′-fluorescent dye-ON-CPG (Toyobo), which is a column containing the acceptor fluorescent dye introduced thereto, in the process of the chemical synthesis of the oligonucleotide by a DNA synthesizer according to the phosphoroamidite method. By using Amino Modifier II (Toyobo), an amino group is directly introduced to the 5′ terminal of the oligonucleotide in a DNA synthesizer, and then amino acylating reaction with an active esterified nano particle fluorescent material can be carried out to label the 5′ terminal of the oligonucleotide with the nano particle fluorescent material.

The nucleotide sequence has a base sequence that is complementary to the target nucleic acid sequence. In order to detect the specific sequence in a normal target nucleic acid, a sequence that is perfectly complementary to the specific sequence is most preferred. However, the degree of complementarity may be changed appropriately depending on the type of the specific sequence or the purpose of the detection.

The nucleotide may be a synthesized nucleotide that is complementary to an arbitrary sequence, a genome extracted from a cell, a fragment obtained by decomposition of any of such nucleotide with a restriction enzyme, or an RNA.

The nucleotide sequence may be complementary to a sequence resulting from the expected specific point mutation in the target specific base sequence. Such a nucleotide enables more accurate detection of the point mutation.

Examples of the probe for detecting point mutation in the target include, but are not limited to, gene p53 related to hepatic carcinoma, gene Ki-ras related to pancreatic carcinoma, and others disclosed in JP-A No. 10-127300, Gene, 70, 245-252 (1988), Nature, 350, 427-428 (1991), Nature, 304, 497-500 (1983), and Am. J. Pathol., 143, 545-554 (1993).

The nucleotide sequence for detecting point mutation preferably has the mutation portion in the central portion of the probe moiety in order to avoid false positive results. The central portion refers to the center or the neighborhood of the center of the nucleotide sequence that constitutes the probe moiety. By this design, the sensitivity at detecting the presence or absence of the mutation can be improved.

In the fluorescent probe according to the invention, in addition to the probe moiety, arm moieties made of nucleotide sequences may be further provided between the donor nano particle fluorescent material and the probe moiety, and between the acceptor dye and the probe moiety. By this design, detection is effective even at the ends of the probe moiety.

The length of each arm moieties is not limited as far as the folding structure in the absence of the target nucleic acid is not impaired. The nucleotide length of each arm moiety is preferably 2 to 20 nucleotides, more preferably 3 to 10 nucleotides, from the viewpoint of the easiness in structural changes upon binding to the target nucleic acid. Preferably, the arm moieties have complementary sequences to each other by which the donor nano particle fluorescent material and acceptor dye are kept close to each other.

In an embodiment, plural fluorescent probes are used simultaneously so as to enable detection of different types of target nucleic acid. In another embodiment, plural sets of probe moiety and acceptor dye are provided for one donor nano particle fluorescent material such that plural target nucleic acids can be detected.

In such a fluorescent probe having plural types of probe moiety and acceptor dye, plural types of acceptor dye corresponding to plural types of probe moiety are used for labeling. When the fluorescent probe is used and a probe moiety capable of forming a duplex with the target nucleic acid is present, only the acceptor dye of the probe moiety that interacts with the target nucleic acid to form the duplex fails to emit light by FRET. As a result, the fluorescence spectrum pattern of the entire fluorescent probe is changed. By comparing the fluorescence spectrum patterns, plural types of target nucleic acid can be detected simultaneously with high sensitivity.

[Target Nucleic Acid]

The target nucleic acid to be detected by the fluorescence probe is not particularly limited in type, length, or form thereof, and both DNA and RNA are suitable. Examples of DNA include DNA derived from biological material, such as extracted and purified DNA, and synthesized DNA. Types of usable DNA include genomic DNA, cDNA, extracted and purified DNA, DNA fragments, and DNA integrated into a vector. Examples of RNA include RNA derived from biological material, such as extracted RNA, and synthesized RNA. Types of usable RNA include mRNA, extracted and purified RNA, and RNA fragments.

Usually, the target nucleic acid that can be detected by the fluorescence probe is a single chain, but the target nucleic acid may be a duplex, or a multiplex having three or more chains. In such a case, the chains of the target nucleic acid are preferably dissociated so as to allow the reaction with the fluorescent probe.

<Fluorescence Detecting Method>

The fluorescence detecting method according to the invention includes placing the fluorescent probe under a condition in which the fluorescent probe can interact with coexisting target nucleic acid (reaction process), and measuring the emission from the fluorescent probe after irradiating the excitation light for the nano particle fluorescent material (emission measuring process).

[Condition for the Reaction of Probe and Target Nucleic Acid]

The condition in which the fluorescent probe can interact with the target probe may be a normal hybridization condition for nucleic acid. In an exemplary condition, 25 nM of the probe and 150 nM of the target nucleic acid are mixed in a solution containing 10 mM Tris-HCl (pH 8.0), 50 mM KCl, and 1.5 mM MgCl₂, followed by leaving the mixture at a constant temperature for about 30 minutes. Of course, the reaction is not limited to this particular condition.

The temperature of the hybridization may be adequately selected according to the length and the base sequence of the probe, the base sequence of the target, and others.

When the target nucleic acid is in the form of a duplex, a process for dissociating the duplex may be provided before the reaction process. The dissociation may be conducted under a condition that is usually employed for dissociating duplex nucleic acid. For example, the duplex may be put in a high temperature condition for dissociation. The temperature for the dissociation may be a temperature that is usually adopted for that purpose, and may be higher than room temperature. The temperature for the dissociation is generally around 100 deg. C., but the temperature is not particularly limited.

The detection of the emission in the emission measuring process may be conducted satisfactorily with an ordinary emission measuring method.

For example, when zinc oxide nano particle is used as the nano particle fluorescent material and HEX is used as the acceptor fluorescent dye, the excitation light near 340 nm is used for excitation, and the fluorescence spectrum within the wavelength range of 500 nm to 650 nm is measured. The range of the wavelength of the excitation light and the fluorescence spectrum to be measured may be selected according to the nano particle fluorescent material to be used and the precision of the light wavelength selection of the measuring instrument. In another embodiment, zinc oxide nano particles are excited by light around 340 nm, and the fluorescence intensity of the zinc oxide nano particles at 520 nm and fluorescence intensity at 556 nm, which is the fluorescence peak wavelength of HEX, are measured simultaneously by being separated into two fluorescent components with a spectroscopic system.

When the nano particle fluorescent material is excited, the fluorescent resonant energy can be transferred to the acceptor dye.

In the fluorescence detecting method according to the invention, when the fluorescence intensity is measured in the emission measuring process, the presence or absence of the specific sequence or point mutation in the target nucleic acid is detected based on the fluorescent intensity obtained. Since the difference in the state of the hybridization between the fluorescent probe and the target nucleic acid is used for detection, any evaluation method capable of evaluating the difference in the state of the hybridization can be suitably employed. For example, the evaluation may be made on the basis of the ratio of the emission degree at the maximum fluorescence wavelength of the acceptor dye to the emission degree at the maximum fluorescence wavelength of the donor nano particle fluorescent material. Of course, other evaluation methods are also usable.

By conducting such evaluation, which is based on the fluorescence intensity, on the results of the measurement in various conditions, it is possible to detect easily not only the presence or absence of the target nucleic acid, but also various mutations in the specific sequence, such as single nucleotide polymorphism, substitution, insertion or deletion of plural bases. Conditions for detecting the mutations include those mentioned above as the hybridization conditions. For example, the temperature and/or the salt concentration can be adjusted.

In the following, an exemplary method for detecting point mutation is described in which the fluorescence detecting method according to the invention is applied to the target nucleic acid obtained from biological material is described. However, the detection method according to the invention is not limited thereto.

Biological material is not specified, and secretion, blood, excrement, or tissue from the subject may be used satisfactorily.

[Detection Method of Point Mutation in Target Nucleic Acid Obtained from Biological Material]

(1) Extraction of Nucleic Acid from Biological Material

Ordinary methods for extracting nucleic acid, such as DNA or RNA, can be suitably used for the extraction of nucleic acid from biological material. For example, the following method may be employed, but the extraction method is not limited thereto. When extracting DNA from blood, the blood is centrifuged for 15 minutes at 1000 rpm, and the precipitate is washed 3 times with PBS. After aqueous components are removed, protein is decomposed by 100 μg/mL of protease K. After the sample is left at 50° C. for 3 hours, an equivalent amount of water saturated phenol is added, and the sample is centrifuged for 10 minutes at room temperature at 3000 rpm. Thereafter, the phenol is removed, and water saturated phenol is added again. The resultant phenol layer is removed by centrifugation, and a double amount of chloroform is added, and the sample is centrifuged for 2 minutes at 3000 rpm. The chloroform treatment is repeated 3 times, and the sample is dialyzed against 100 times as much volume of a solution of 50 mM-Tris-HCl (pH 7.4), 10 mM EDTA, and 10 mM NaCl for several hours. Then, 20 μg/mL of RNaseA is added, the sample is held at 37° C. for 30 minutes, and the above phenol-chloroform treatment is repeated.

(2) Amplification by PCR

A portion of a gene containing base sequence that possibly has mutation is amplified. For example, the portion can be amplified by the PCR method using two primers flanking the region of the specific sequence that is expected to include mutation. The amplification by PCR may be suitably conducted by a method that is employed in usual PCR methods as described in Methods Enzymol. (1987), Vol. 15, No.5, 335-350. When the target nucleic acid is RNA, DNA is first synthesized from RNA by using a reverse transcriptase, and then the DNA can be amplified by the PCR method (See Mol. Cell. Biol., (1987), 7, 3231-3236).

As the control, a nucleic acid having the normal base sequence is prepared by synthesis or the like.

(3) Hybridization Between Probe and Target Nucleic Acid

In a solution of a target nucleic acid amplified by the method above or in a solution of the normal nucleic acid as the control, the labeled fluorescent probe according to the invention is added, and reaction is allowed to proceed in the hybridization condition described above.

(4) Measurement of Ratio of Fluorescence Intensity (FRET Efficiency)

The fluorescence spectrum is measured with a fluorescence spectrophotometer at an adequate predetermined temperature selected according to the probe to be used.

Based on the fluorescence spectrum measured when the donor nano particle fluorescent material is excited by light, the emission degree ratio (Y/X) is determined, that is, the ratio of the emission degree (X) at the maximum emission wavelength (Anm) of the nano particle fluorescent material to the emission degree (Y) at the maximum emission wavelength (Bnm) of the acceptor dye. The presence of absence of point mutation in the sample nucleic acid can be determined by the comparison between the result obtained when the sample nucleic acid derived from biological material is used and the result obtained when the normal nucleic acid is used.

When the method according to the invention is used for measuring biological sample, the sample may be a biological tissue or a nucleic acid extracted from biological material. When a biological tissue is used, the biological sample is fixed on a support (such as a plate), the target DNA portion is amplified by PCR method, a reaction solution containing the probe according to the invention is added, the temperature is raised, the excitation light is irradiated, and the emitted fluorescence is measured. When using the nucleic acid extracted from biological material, a reaction solution containing the probe according to the invention is added to a solution containing the sample nucleic acid that has optionally been amplified, the temperature is raised, the excitation light is irradiated, and the emitted fluorescence is measured.

Exemplary embodiments of the invention are described below.

(1) A fluorescent probe having a base sequence complementary to a specific sequence in a target nucleic acid, the fluorescent probe having one end labeled with a nano particle fluorescent material, and the other end labeled with a fluorescent dye capable of fluorescence resonance energy transfer from the nano particle fluorescent material.

(2) The fluorescent probe according to (1), wherein the number-average particle diameter of the nano particle fluorescent material is from 0.5 to 100 nm.

(3) The fluorescent probe according to (1) or (2), wherein the nano particle fluorescent material is a metal oxide or metal sulfide whose surface is modified with a surface modifier, and the surface modifier is a compound represented by formula (I) or a decomposition product thereof.
M-(R)₄ (I)

In formula (I), M represents a Si or Ti atom, and R represents an organic group. Rs may be the same as or different from each other, and at least one of Rs represents a group that is reactive to an affinity molecule.

(4) The fluorescent probe according to (3), wherein the nano particle fluorescent material is an oxide of a metal selected from the group consisting of Y, Eu, Th, Tm, Ba, Ca, Mg, Al, Mn, Zn, Si, Sr, Ga, Yb, Cr, Ce, Pb and W.

(5) The fluorescent probe according to (4), wherein the nano particle fluorescent material is zinc oxide.

(6) The fluorescent probe according to any one of (1) to (5), wherein the fluorescent probe has a base sequence composed of 10 to 70 nucleotides.

(7) The fluorescent probe according to any one of (1) to (6), wherein the base sequence in the fluorescent probe includes a probe moiety complementary to the target nucleic acid, and arm moieties which are connected respectively to the two ends of the probe moiety and which are complementary to each other.

(8) A fluorescence detecting method for detecting a specific sequence in a target nucleic acid with a fluorescent probe having a base sequence complementary to the specific sequence in a target nucleic acid, the method comprising putting the fluorescent probe in a condition in which the fluorescent probe is capable of interacting with the target nucleic acid, irradiating excitation light for a nano particle fluorescent material, and measuring the light emission from the fluorescent probe. The fluorescent probe has one end labeled with the nano particle fluorescent material, and the other end labeled with a fluorescent dye capable of fluorescence resonance energy transfer from the nano particle fluorescent material.

(9) The fluorescence detecting method according to (8), further comprising detecting a point mutation in the specific sequence in the target nucleic acid based on results of the measurement.

(10) The fluorescence detecting method according to (8) or (9), wherein a portion corresponding to a point mutation in the target nucleic acid is present in a central portion of the complementary base sequence in the fluorescent probe.

The invention will be specifically described below by reference to Examples, but the Examples should not be construed as limiting the invention.

### EXAMPLES

### Example 1

### Synthesis of Zinc Oxide Nano Particles

Dehydrated ethanol (250 mL) was added to zinc acetate dihydrate (5.49 g, 25 mmol), and the mixture was mildly heated and refluxed in a Dean-Steark dehydrating apparatus for 2 hours while the solvent was distilled away. The amount of the removed solvent was 150 mL. 150 mL of dehydrated EtOH was added again to the resultant turbid reaction solution, and the solution was heated and refluxed. The reaction solution that became transparent was cooled to room temperature with water.

To the reaction solution, tetramethyl ammonium hydroxide (25% methanol solution,1 1.4 mL, 28 mmol) was added and the mixture was stirred for 4 hours at room temperature. Then, 3-amino propyl trimethoxy silane (4.7 mL, 25 mmol) and water (1.5 mL, 83.3 mmol) were added thereto, and the resultant mixture was stirred for 4 hours at 60° C. 7 minutes after the start of the reaction, a white solid matter deposited. The reaction solution was cooled to room temperature with water, and the solid matter was subjected to suction filtration, and then washed with ethanol. The obtained white powder was dried in reduced pressure to obtain zinc oxide nano particles with aminated surface with a yield of 6.0 g.

The particles (200 mg) were dissolved in 10 mL of distilled water, and were subjected to gel filtration using a column filled with Sephadex G25. In all of the following reactions, this desalinated aqueous solution is used.

The nano particles synthesized according to this prescription show transparent favorable dispersed state even at a concentration of 10 wt. %. The peak pattern thereof coincided with the powdery sample of zinc oxide of hexagonal system (wurtzite) belonging to the X-ray diffraction space group P6₃mc, and the number-average particle diameter thereof was 3 nm when measured with a TEM.

Hereinafter, the concentration of zinc oxide particles was estimated from the volume (14.13×10⁻²⁷ m³mol⁻¹), molecular weight (81.38) and density (5.67) of zinc oxide having a number-average particle diameter of 3 nm.

### Example 2

### Synthesis of Probe Oligonucleotide

The probe oligonucleotides having the following sequences were manufactured by solid phase synthesis using a known phosphoamidite method.

HEX is a fluorescent dye having an absorption maximum wavelength of 534 nm and a fluorescence maximum wavelength of 556 nm. In the sequences, the underline indicates the mutation location.

The probe oligonucleotides were purified by reverse phase column chromatography, and the structures thereof were confirmed by MALDI-TOF mass spectrum.

TABLE 1

(a) Hairpin loop DNA

(Seq. ID No. 1)

5′-H₂N-(C₆)-
ACACGCTCATCATAACCTTCAGCAAGCTTTAACTCATAGTGAGCGTGT-
HEX-3′
(b) Target DNA

(Seq. ID No. 2)
5′-ACGCTCACTATGAGTTAAAGCTTGCTGAAGG-TTATGA-3′
(c) Single nucleotide polymorphism target DNA
(Seq. ID No. 3)

5′-ACGCTCACTATGAGTTCAAG-CTTGCTGAAGGTTATGA-3′

### Example 3

### Binding Zinc Oxide Nano Particles to Oligonucleotide

Zinc oxide nano particles modified with amino groups prepared in Example 1 were dispersed in a solution containing 200 mM of NaCl and 50 mM of HEPES (pH 8) to give a concentration of 150 μM. Succinic anhydride in 10 times as much mole as the zinc oxide particles was added, and the mixture was stirred for 1 hour at room temperature. Then, the mixture was subjected to ultrasonication for 15 minutes, whereby carboxyl groups were introduced onto the particle surface. Excessive succinic anhydride was removed by gel filtration or centrifugal separation.

Thereafter, the oligonucleotide prepared in example 2 (a), 5 equivalents of N-hydroxy succinimide, and WSC were added thereto, and the reaction was allowed to proceed for 3 hours at room temperature, so that a probe having the donor nano particle fluorescent material linked to the 5′ terminal end and the acceptor dye to the 3′ terminal end was obtained.

### Example 4

### Hybridization with Target DNA

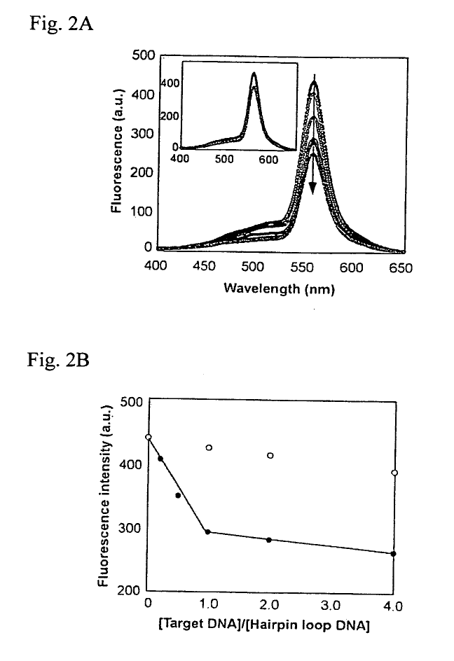

Using the probe of Example 3, and the target DNA prepared in Example 2 (b) and (c), hybridization was carried out at 20° C. in a buffer solution containing 200 mM of NaCl and 50 mM of HEPES (pH 8.0). The change in the fluorescence spectrum(excitation light; 340 nm) observed when the concentration of the target DNA was changed from 0 to 1.2 μM is shown in FIGS. 2A and 2B The main graph in FIG. 2A shows the fluorescence spectrum observed when the full-matching target DNA is used for the hybridization. The framed graph shows the fluorescence spectrum observed when the target DNA including mismatch is used for the hybridization. FIG. 2B shows the change in fluorescence intensity at 560 nm. The solid dots indicate the data obtained when the full match DNA is used, and the blank dots indicate the data obtained when the mismatch DNA is used.

As shown in FIGS. 2A and 2B, the fluorescence intensity is lowered (see the main graph of FIG. 2A and FIG. 2B) by the increase in the amount of the full-matching target DNA. In contrast, the fluorescence intensity is not changed significantly by the increase in the amount of the target DNA including mismatch (see the framed graph of FIG. 2A).

Therefore, when the target DNA fully matched the hairpin sequence of the probe DNA, the fluorescence of the acceptor dye induced by the FRET is decreased. In contrast, it is clear that the presence of single nucleotide polymorphism significantly suppressed decline of the fluorescence intensity. The fluorescent probe of the Example does not require filter or the like for the detection of the target DNA, and is able to detect with high sensitivity even a single nucleotide polymorphism based on the difference from full-matched DNA in the change of the fluorescence intensity.

### Comparative Example

A probe was prepared in the same manner as in Example 2 except that the zinc oxide nano particle fluorescent material as the donor used in Example 2 was changed to fluorescein. A hybridization experiment was conducted in the same manner as in Example 3, but no significant difference was observed between the presence and absence of the single nucleotide polymorphism within the concentration range of the target DNA employed in Example 3.

Therefore the present invention can provide a fluorescent probe capable of detecting a specific nucleic acid sequence with high quantifiability and reproducibility without loss of sensitivity, and a method for detecting a nucleic acid sequence using the same.

What is claimed is:

1. A fluorescent probe comprising a base sequence complementary to a specific sequence in a target nucleic acid, wherein the fluorescent probe has one end labeled with a nano particle fluorescent material, and the other end labeled with a fluorescent dye capable of fluorescence resonance energy transfer from the nano particle fluorescent material.

2. The fluorescent probe according to claim 1, wherein a number-average particle diameter of the nano particle fluorescent material is from 0.5 to 100 nm.

3. The fluorescent probe according to claim 1, wherein a number-average particle diameter of the nano particle fluorescent material is from 1 to 10 nm.

4. The fluorescent probe according to claim 1, wherein the nano particle fluorescent material is a metal oxide or a metal sulfide.

5. The fluorescent probe according to claim 1, wherein the nano particle fluorescent material is a metal oxide selected from Y, Eu, Tb, Tm, Ba, Ca, Mg, Al, Mn, Zn, Si, Sr, Ga, Yb, Cr, Ce, Pb or W.

6. The fluorescent probe according to claim 4, wherein the nano particle fluorescent material is zinc oxide.

7. The fluorescent probe according to claim 1, wherein the nano particle fluorescent material is a metal oxide or metal sulfide whose surface is modified with a surface modifier, and the surface modifier is a compound represented by formula (I) or a decomposition product thereof:
M-(R)₄ (I)
wherein, in formula (I), M represents a Si or Ti atom; R represents an organic group; Rs may be the same as or different from each other; and at least one of Rs represents a group that is reactive to an affinity molecule.

8. The fluorescent probe according to claim 7, wherein the group that is reactive to an affinity molecule has, at a terminal thereof, a vinyl group, an allyloxy group, an acryloyl group, a methacryloyl group, an isocyanato group, a formyl group, an epoxy group, a maleimide group, a mercapto group, an amino group, a carboxyl group, or a halogen.

9. The fluorescent probe according to claim 7, wherein the group that is reactive to an affinity molecule has an amino group at a terminal thereof.

10. The fluorescent probe according to claim 1, wherein the fluorescent probe has a base sequence composed of 10 to 70 nucleotides.

11. The fluorescent probe according to claim 1, wherein the base sequence of the fluorescent probe includes a probe moiety complementary to the target nucleic acid, and arm moieties which are connected respectively to the two ends of the probe moiety and which are complementary to each other.

12. The fluorescent probe according to claim 11, wherein each arm moiety is composed of 2 to 20 nucleotides.

13. The fluorescent probe according to claim 11, wherein a portion corresponding to a point mutation in the target nucleic acid is present in a central portion of the complementary base sequence in the fluorescent probe.

14. A fluorescence detecting method for detecting a specific sequence in a target nucleic acid with a fluorescent probe comprising a base sequence complementary to the specific sequence in the target nucleic acid, the method comprising:
putting the fluorescent probe in a condition in which the fluorescent probe is capable of interacting with the target nucleic acid;
irradiating excitation light for a nano particle fluorescent material; and
measuring the light emission from the fluorescent probe,
wherein the fluorescent probe has one end labeled with the nano particle fluorescent material, and the other end labeled with a fluorescent dye capable of fluorescence resonance energy transfer from the nano particle fluorescent material.

15. The fluorescence detecting method according to claim 14, further comprising detecting a point mutation in the specific sequence in the target nucleic acid based on results of the measurement.

16. The fluorescence detecting method according to claim 14, wherein a portion corresponding to a point mutation in the target nucleic acid is present in a central portion of the complementary base sequence in the fluorescent probe.

17. The fluorescence detecting method according to claim 14, wherein the excitation light is ultraviolet light having a wavelength of 250 nm to 380 nm.


SEQUENCE LISTING (3 sequences)

SEQ ID NO: 1 - DNA, 48 bp, Artificial Sequence
  hair pin loop probe
acacgctcatcataaccttcagcaagctttaactcatagtgagcgtgt

SEQ ID NO: 2 - DNA, 37 bp, Artificial Sequence
  Target DNA
acgctcactatgagttaaagcttgctgaaggttatga

SEQ ID NO: 3 - DNA, 37 bp, Artificial Sequence
  SNPs target DNA
acgctcactatgagttcaagcttgctgaaggttatga